(19) United States
(12) Patent Application Publication
(10) Pub. No.: US 2023/0101409 A1
(43) Pub. Date: Mar. 30, 2023

(54) ANTI-COUNTERFEIT TAGS USING HIGH-COMPLEXITY POLYNUCLEOTIDES

(71) Applicant: MICROSOFT TECHNOLOGY LICENSING, LLC, Redmond, WA (US)

(72) Inventors: Yuan-Jyue CHEN, Seattle, WA (US); Bichlien Hoang NGUYEN, Seattle, WA (US); Jake Allen SMITH, Seattle, WA (US); Karin STRAUSS, Seattle, WA (US)

(21) Appl. No.: 17/490,615

(22) Filed: Sep. 30, 2021

(51) Int. Cl. G16B 30/10; C12Q 1/6876; G06K 7/10

(52) U.S. Cl. CPC G16B 30/10; C12Q 1/6876; G06K 7/10366

(57) ABSTRACT

Large numbers of polynucleotides with random sequences are used collectively as a molecular anti-counterfeiting tag. The polynucleotides are sequenced, placed on an item, and the sequences stored in an electronic record. Authenticity is determined by collecting the polynucleotides from a labeled item, sequencing those polynucleotides, and comparing the sequence to that stored in the electronic record. The number of polynucleotides used as the tag may be adjusted by aliquoting the original batch of randomly synthesized polynucleotides. Complexity of the polynucleotide tags may be increased by assembling individual polynucleotides from multiple dilutions to create longer assembled polynucleotides. Even if the sequences of the polynucleotides are known, the complexity of the tag can make the forgery of the tag itself technically difficult and prohibitively expensive.

### BIOLOGICAL SEQUENCES

Although this application references nucleotide sequences and uses single-letter abbreviations to represent individual nucleic acid bases, it does not include any nucleotide sequences as defined in 37 C.F.R. 1.821 because there are no sequences of ten or more nucleotides.

### BACKGROUND

Forgeries and counterfeits are problems in many industries and for many types of items. Purchasers of unique, high-value items such as artwork may insist on verification of authenticity. Identifying a forgery or counterfeit item can be challenging because of the difficulty of tracking provenance over time and through long supply chains. Authenticity may be attested to by an expert, but that requires trusting the expert's skill and sincerity. One solution is to use a label or tag rather than characteristics of the item itself to signal authenticity. Anti-counterfeit tags can be used to make authentic items distinguishable from counterfeit or fake items. An anti-counterfeit tag is placed on an item and absence of the correct tag indicates an inauthentic item. Holographic stickers, radio-frequency identification (RFID) tags, and quick response (QR) codes are all used as anti-counterfeit tags.

However, many types of anti-counterfeit tags can themselves be forged by sophisticated bad actors. The problem is especially acute for high-value items in which the potential profit greatly exceeds the cost of making a fake tag. Accordingly, it is desirable to develop new types of anti-counterfeit tags that are relatively easy and inexpensive to produce and validate but difficult and expensive to copy. The following disclosure is made with respect to these and other considerations.

### SUMMARY

This disclosure provides techniques for creating and using polynucleotides as anti-counterfeit tags. Instead of using a single polynucleotide as a tag, a large number such as millions, billions, hundreds of billions, trillions, or more of polynucleotides each with a unique, random sequence of bases are used to tag an item. Column synthesis of polynucleotides can create numbers of unique molecules on the order of 10²⁴ for each synthesis. The polynucleotides are synthesized by a process that creates a batch of individual polynucleotide strands each with a different, random sequence of bases. Many different polynucleotide strands each with random sequences (or “random-mers”) can be synthesized in a single batch for about the same cost as synthesizing multiple copies of a polynucleotide with a single, specific base sequence. The techniques of this disclosure take advantage of this cost difference to create anti-counterfeit tags that are much less expensive to generate than to copy.

The polynucleotides are sequenced, and the sequences are stored in an electronic record such as a cloud database. The electronic record associates the sequences with a description of the tagged item such as a picture or textual description. The electronic record may be maintained by a trusted third party and serves as an objective source for validating the authenticity of the tagged item. The sequences in the electronic record may be publicly available. The synthetic polynucleotides with random sequences are then placed on the item.

The number of polynucleotides used as a tag may be adjusted by taking a random subset from the collection of polynucleotides synthesized with random sequences. One way of creating a random subset is by dividing or taking an aliquot from the batch of synthesized polynucleotides. Using only a subset of the polynucleotides as the anti-counterfeit tag reduces the sequencing cost for characterizing the tag.

To further increase the complexity of the polynucleotide tag, multiple polynucleotides may be joined together by an assembly technique such as Gibson assembly, golden gate assembly, or overlap-extension polymerase chain reaction. Assembled polynucleotides are created from two or more of the random subsets taken from the original batch of synthetic polynucleotides. The assembly technique may join polynucleotides from different random subsets in a specific order. Each assembled polynucleotide will have a different sequence than the other assembled polynucleotides. This increases the diversity of sequences without synthesizing additional polynucleotides and without increasing the total number of molecules that must be sequenced to validate the authenticity of an item. The cost for some sequencing technologies, such as nanopore sequencing, is affected more by the number of molecules that are sequenced rather than the length of each molecule. Thus, sequencing the same number of longer molecules may not increase the cost of sequencing. Joining multiple shorter polynucleotides together in an assembled polynucleotide also creates a polynucleotide that is longer than the maximum length that can be accurately synthesized by current chemical synthesis techniques. This further increases the cost and difficulty of forging the polynucleotide tag.

Authenticity of an item is determined by collecting polynucleotides from the item and sequencing the polynucleotides. Sequencing may be performed by any sequencing technique such as, but not limited to, nanopore sequencing. The sequences of the polynucleotides are provided to a computing device connected to the electronic record and compared to the stored sequences. If there is a match, an indication of authenticity is returned. To reduce the sequencing cost of validating an item, fewer than all the polynucleotides collected may be sequenced and compared to the electronic record.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The figures are schematic representations and items shown in the figures are not necessarily to scale.

FIG. 1 illustrates use of synthetic polynucleotides with random sequences and an electronic record to validate the authenticity of an item.

FIG. 2A illustrates steps for creating an assembled polynucleotide.

FIG. 2B illustrates additional random end sequences on the ends of synthetic polynucleotides that also include nonrandom sequences.

FIG. 3A illustrates an entry in an electronic record used for determining the authenticity of an item tagged with polynucleotides.

FIG. 3B is a Venn diagram showing sets of sequences that may be used for determining if an item is authentic.

FIG. 4 is a flow diagram showing an illustrative process for using polynucleotides as an anti-counterfeit tag.

FIG. 5 is an illustrative computer architecture for implementing techniques of this disclosure.

### DETAILED DESCRIPTION

There are few choices for anti-counterfeit tags that can be directly applied to an item, are relatively easy for a potential purchaser to verify, and are difficult for a bad actor to forge. High-complexity polynucleotide tags have all these characteristics.

Nucleic acids have been previously identified as taggants in U.S. Pat. No. 5,451,505. However, the '505 patent and other previous work discussing polynucleotide tags use the sequence of one or a few polynucleotides as the tag. Due to advances in polynucleotide synthesis and sequencing technology, simple polynucleotide tags can now be readily copied by a bad actor if the sequence is known. Copying of the polynucleotide tag itself may be prevented by keeping the existence and sequence of the tag secret. However, keeping the tag secret prevents a purchaser or potential purchaser from independently confirming the authenticity of the item. Thus, new designs and techniques for polynucleotide tags are needed so that purchasers can independently verify the authenticity of an item while preventing copying of the polynucleotide tags themselves by a bad actor.

Detail of procedures and techniques not explicitly described or other processes disclosed of this application are understood to be performed using conventional molecular biology techniques and knowledge readily available to one of ordinary skill in the art. Specific procedures and techniques may be found in reference manuals such as, for example, Michael R. Green & Joseph Sambrook, Molecular Cloning: A Laboratory Manual, Cold Spring Harbor Laboratory Press, 4th ed. (2012).

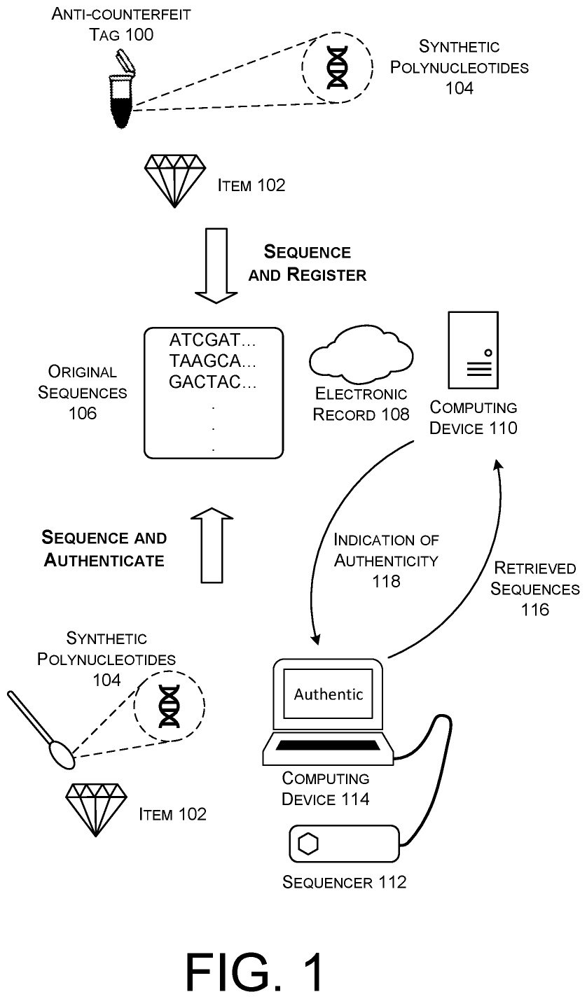

FIG. 1 shows the use of an anti-counterfeit tag 100 to label and identify an item 102. The anti-counterfeit tag 100 contains a large number of synthetic polynucleotides 104 with random sequences. The plurality of polynucleotides 104 rather than any single polynucleotide functions as the anti-counterfeit tag 100. Thus, reproducing the anti-counterfeit tag 100 will require sequencing all of the synthetic polynucleotides 104. Polynucleotides include both deoxyribonucleic acid (DNA), ribonucleic acid (RNA), and hybrids containing mixtures of DNA and RNA. DNA and RNA include nucleotides with one of the four natural bases cytosine (C), guanine (G), adenine (A), thymine (T), or uracil (U) as well as unnatural bases, noncanonical bases, and modified bases. The synthetic polynucleotides 104 may be double-stranded polynucleotides such as in one implementation double-stranded DNA. The synthetic polynucleotides 104 have non-natural sequences that are not derived from natural or biological sources.

Multiple techniques for synthesizing polynucleotides with random sequences are known to those of ordinary skill in the art and polynucleotides with random sequences can be ordered from commercial suppliers. See Meiser, Koch, J., Antkowiak, P. L. et al. DNA synthesis for true random number generation. Nat Commun 11, 5869 (2020), The enzyme terminal deoxynucleotidyl transferase (TDT) used in enzymatic polynucleotide synthesis is known to generate random sequences. See Fowler J D, Suo Z (2006) Biochemical, Structural, and Physiological Characterization of Terminal Deoxynucleotidyl Transferase. Chemical Reviews 106(6):2092-2110. In these techniques, the base added to any given strand at any round of synthesis is determined stochastically leading to synthesis of polynucleotides with random sequences. If there is unequal incorporation of different nucleotides, the reaction conditions may be adjusted so that each nucleotide has an equal probability of being incorporated at each strand during each round of addition (e.g., 25% chance for each of A, G, C, and T). If double-stranded polynucleotides are used for the anti-counterfeit tag 100, strands complementary to the synthesized polynucleotides may be created by polymerase chain reaction (PCR) to form double-stranded molecules.

One technique for creating a large number of polynucleotides with random sequences is column synthesis using the phosphoramidite method. During column synthesis of random polynucleotides, individual nucleosides are mixed prior to entering a solid state binding substrate, where they start forming a polynucleotide strand based on their coupling efficiencies. The rate of the individual nucleotides couplings, rᵢ, can be approximated by multiplication of the respective rate constant, kᵢ and the nucleotide concentration, cᵢ. During the process, individual nucleotides are shielded from binding to other nucleotides using protecting groups, ensuring that only one new random nucleotide can bind per polynucleotide strand per iteration. Excess nucleotides that have not found a polynucleotide strand to bind to are then removed from the synthesis chamber, and polynucleotide strands are de-protected. To elongate each polynucleotide strand to the desired length, the process of adding a mix of nucleotides, washing off left-over and subsequently de-protecting is repeated as often as required. Once the desired strand length of polynucleotides has been reached, the polynucleotides are cleaved from the synthesis support.

The polynucleotides used for an anti-counterfeit tag 100 may also be created by randomly fragmenting genomic DNA. Techniques such as shearing that break genomic DNA into shorter strands are known to those of ordinary skill in the art. The locations where the genomic DNA is broken are not known in advance, however, the actual sequences are not generated randomly. Thus, in some implementations, fragments of genomic DNA or of natural DNA may be used as the anti-counterfeit tag 100 instead of synthetic random polynucleotides.

The anti-counterfeit tag 100 may contain a large number of synthetic polynucleotides 104 such as from about 10² to about 10²⁴ different polynucleotides or more. For example, an anti-counterfeit tag 100 may contain about 10¹², 10¹⁸, or 10²⁴ polynucleotide strands each with different, random sequences. There is a very small possibility that two or more of the randomly generated polynucleotides will have the same sequence. However, For practical purposes, it can be assumed that all the polynucleotides synthesized in one batch and used as an anti-counterfeit tag 100 have different sequences.

The synthetic polynucleotides 104 are placed on an item 102. The item 102 may be a high-value item such as a work of art, a jewel, a banknote, a document, an antique, etc. The synthetic polynucleotides 104 may be placed directly on the surface of the item 102 for example in liquid or power form. If the item 102 itself is liquid, the synthetic polynucleotides 104 may be mixed into the item 102. The synthetic polynucleotides 104 may be applied “naked” without any modification or they may be protected with stabilizing agents or encapsulated by a protective coating. Multiple techniques for stably storing polynucleotides have been developed for storing biological samples and are known to those of ordinary skill in the art. Any suitable technique may be adapted for use with the item 102 depending on the composition of the item 102. In some implementations, the synthetic polynucleotides 104 may be placed on, under, or in a second taggant that is visibly detectable such as a QR code, RFID tag, or holographic sticker.

Because the synthetic polynucleotides 104 are synthesized by a process that creates random sequences, the sequences of the synthetic polynucleotides 104 are not known in advance of synthesis. Following synthesis, and before application to the item 102, the synthetic polynucleotides 104 are sequenced. All polynucleotides intended to be used as a tag should preferably be sequenced at least initially in order to characterize the tag. However, later verification of a tag could use the sequences of less than all the polynucleotides depending on the desired level of confidence.

Some techniques for synthesizing multiple polynucleotides with random sequences create only one copy of each sequence. And most sequencing procedures discard the polynucleotide strands following sequencing. Accordingly, to both sequence a synthetic polynucleotide 104 with a random sequence and to also place a polynucleotide strand with the same sequence on an item 102 as an anti-counterfeit tag 100 there may need to be multiple copies of each polynucleotide strand.

The synthesized polynucleotide strands may be copied to generate multiple copies. Any technique that creates multiple copies of an existing polynucleotide strand may be used. Current techniques known to those of ordinary skill in the art for making multiple copies of existing polynucleotide strands include enzymatic methods. One enzymatic technique to exponentially amplify polynucleotides is the well-known PCR. Isothermal amplification methods are another enzymatic technique. Isothermal methods typically employ unique DNA polymerases for separating duplex DNA. Isothermal amplification methods include Loop-Mediated Isothermal Amplification (LAMP), Whole Genome Amplification (WGA), Strand Displacement Amplification (SDA), Helicase-Dependent Amplification (HDA), Recombinase Polymerase Amplification (RPA), and Nucleic Acid Sequences Based Amplification (NASBA). See Yongxi Zhao, et al., Isothermal Amplification of Nucleic Acids, Chemical Reviews, 115 (22), 12491-12545 (2105) for a discussion of isothermal amplification techniques.

PCR refers to a reaction for the in vitro amplification of specific DNA sequences by the simultaneous primer extension of complementary strands of DNA. In other words, PCR is a reaction for making multiple copies or replicates of a target nucleic acid flanked by primer binding sites. The reaction comprising one or more repetitions of the following steps: (i) denaturing the target nucleic acid, (ii) annealing primers to the primer binding sites, and (iii) extending the primers by a template-dependent polymerase in the presence of nucleoside triphosphates. Usually, the reaction is cycled through different temperatures optimized for each step in a thermocycler. A thermocycler (also known as a thermal cycler, PCR machine, or DNA amplifier) can be implemented with a thermal block that has holes where tubes holding an amplification reaction mixture can be inserted. Other implementations can use a microfluidic chip in which the amplification reaction mixture moves via a channel through hot and cold zones.

Each cycle doubles the number of copies of the specific DNA sequence being amplified. This results in an exponential increase in copy number. Particular temperatures, durations at each step, and rates of change between steps depend on many factors well-known to those of ordinary skill in the art, e.g., exemplified by the references: McPherson et al., editors, PCR: A Practical Approach and PCR 2: A Practical Approach (IRL Press, Oxford, 1991 and 1995, respectively). Illustrative methods for detecting a PCR product using an oligonucleotide probe capable of hybridizing with the target sequence or amplicon are described in Mullis, U.S. Pat. Nos. 4,683,195 and 4,683,202; EP No. 237,362.

However, creating multiple copies of the synthetic polynucleotides 104 is not necessary in some implementations. Polynucleotide strands may be recovered following most sequencing procedures even though they are typically discarded. Thus, it is possible to generate only one copy of each of the synthetic polynucleotides with random sequences, sequence those polynucleotide strands, recover the polynucleotide strands following sequencing, and place the same molecules that were sequenced on the item 102 as the anti-counterfeit tag 100. Moreover, future sequencing technologies may not discard the polynucleotide strands following sequencing (e.g., in situ sequencing).

At least some of the synthetic polynucleotides 104 are sequenced. As described above, a subset of the synthetic polynucleotides 104 following the creation of multiple copies of each of the synthetic polynucleotides, may be used for sequencing. This subset may include a sufficiently sized sample that, given the number of copies of each unique polynucleotide strand and the concentration of the polynucleotide strands, there is a high probability of containing at least one copy of each unique polynucleotide strand. There may be a nearly 100% probability that the subset contains unique polynucleotides strands that represent some percentage (e.g., 99.9%, 99%, 95%, or 90%) of the total number of unique polynucleotide strands that were synthesized.

Sequencing may be performed by any current or later-developed technique for polynucleotide sequencing such as sequencing-by-synthesis or nanopore sequencing. Techniques for sequencing polynucleotides are well known to those of ordinary skill in the art. Sequences of the synthetic polynucleotides 104 of the anti-counterfeit tag 100 are referred to as original sequences 106. The original sequences 106 refer to a representation of the nucleotide bases in the synthetic polynucleotides 104 such as, for example, an electronic file containing text strings of single-letter representations of nucleotide bases (i.e., A, G, C, and T). As discussed above, there may be some synthetic polynucleotides 104 that are not sequenced and thus are not represented in the original sequences 106. However, in most implementations essentially all of the synthetic polynucleotides 104 will be sequenced and included in the original sequences 106. Thus, there may be essentially the same number of sequence strings in original sequences 106 as the number of synthetic polynucleotides 104 with unique random sequences.

The original sequences 106 are transmitted to an electronic record 108. This may be referred to as registering the sequence of the anti-counterfeit tag 100. The electronic record 108 may be a database or other system for storing and organizing electronic data. In some implementations, the electronic record 108 may be maintained by one or more computing devices 110 that are physically distant from the polynucleotide sequencer that generated the original sequences 106 and physically distant from the item 102. For example, the electronic record 108 may be maintained by a network server or in a “cloud” implementation maintained in redundant format by multiple different pieces of hardware connected to a network such as the Internet. The electronic record 108 may be maintained by a third party that is not directly involved in any transactions with the item 102.

The original sequences 106 stored in the electronic record 108 may be publicly available. Thus, anyone can access and read or download the original sequences 106. This makes it possible for anyone to validate the authenticity of the item 102 but also provides a bad actor with the information needed to create a copy of the anti-counterfeit tag 100. However, the large number of synthetic polynucleotides 104 make copying expensive. Although the synthetic polynucleotides 104 were created by a process that generates random sequences, those same sequences cannot be regenerated by another random synthesis. To recreate the synthetic polynucleotides 104, a bad actor would have to perform one synthesis run for each of the thousands, millions, or even billions of unique random sequences included in the synthetic polynucleotides 104. For example, it may cost the legitimate creator of the anti-counterfeit tag 100 about $9 to synthesize 1 trillion polynucleotides with random sequences but it would cost $9×1 trillion for a total of $9 trillion to synthesize each of those unique sequences individually. While parallelized, array-based polynucleotide synthesis is capable of decreasing the per-strand cost, modern techniques produce on the order of 1 million unique polynucleotides per parallelized synthesis. Even with this scaling considered, it would still require a cost premium on the order of a million times to counterfeit the pool of 1 trillion polynucleotides considered above. Thus, it may be prohibitively expensive for a bad actor to use de novo synthesis to reproduce a large number of synthetic polynucleotides 104 with the same random sequences.

The authenticity of the item 102 can be determined by collecting the synthetic polynucleotides 104 from the item 102. If the synthetic polynucleotides 104 of the anti-counterfeit tag 100 are placed on a specific location on the item 102, that location may also be included in the electronic record 108 to guide collection of the polynucleotides 104. The synthetic polynucleotides 104 may be collected from the item 102 by swabbing the surface, removing a portion of the item 102 and extracting the polynucleotides, rinsing the item 102 and extracting the polynucleotides from the rinse solution, or by another technique. Many techniques and commercial kits for collecting, purifying, preparing samples for sequencing are known to those of ordinary skill in the art. For example, techniques developed for environmental or forensic samples may be used to collect and process the synthetic polynucleotides 104 collected from the item 102. See Hinlo R., Gleeson D., Lintermans M., Furlan E. (2017) Methods to maximise recovery of environmental DNA from water samples. PLoS ONE 12(6) and Butler, John M. Forensic DNA Typing—Biology, Technology, and Genetics of STR Markers” Second Edition, Elsevier Academic Press, Burlington, Mass. (2005).

The synthetic polynucleotides 104 collected from the item 102 are provided to a sequencer 112 and sequenced. In some implementations, the synthetic polynucleotides 104 may be processed by techniques known to those of ordinary skill in the art to prepare the sample for sequencing. For example, the polynucleotides collected from the item 102 may be cleaned or have impurities removed. The number of copies of the synthetic polynucleotides 104 may be further increased by techniques such as PCR. The sequencer 112 may be any type of device that can detect the nucleotide base sequence of polynucleotides.

In some implementations, only a portion of the synthetic polynucleotides 104 is sequenced. Sequencing only a portion of the synthetic polynucleotides 104 may be intentional or unintentional. For example, recovering the synthetic polynucleotides 104 from item 102 may fail to collect all of the synthetic polynucleotides 104 applied to the item.

Sequencing fewer than all of the synthetic polynucleotides 104 collected from the item 102 results in a lower sequencing cost while still providing validation of authenticity. For example, a subsample of the synthetic polynucleotides 104 collected from item 102 may be used for sequencing without sequencing the remainder of the sample. The portion of the synthetic polynucleotides 104 may be selected randomly such as by taking an aliquot of the polynucleotides. A bad actor will not be able to know which of the synthetic polynucleotides are 104 selected for sequencing so forging the anti-counterfeit tag 100 will still require synthesis of the entire set of synthetic polynucleotides 104. As used herein, a portion of the synthetic polynucleotides 104 can mean fewer than 1%, about 1%, fewer than 10%, or about 10% of the total number of synthetic polynucleotides 104 recovered from the item 102. A substantial portion of the synthetic polynucleotides 104 means at least about 50% of the total number of synthetic polynucleotides 104 recovered from the item 102. A portion of the synthetic polynucleotides 104 is more than one polynucleotide and may include at least 100 polynucleotides, at least 1,000 polynucleotides, at least 10,000 polynucleotides, or at least 100,000 polynucleotides. In some implementations, the size of the portion (and thus the cost of sequencing) may be based on a value of the item 102. For example, the size of the portion may be selected such that the cost of sequencing is about 0.01%, about 0.1%, about 0.5%, about 1%, about 2%, about 3%, about 4%, or about 5% of the value of the item.

In some implementations, the sequencer 112 may be a nanopore sequencer. Nanopore sequencing reads the sequence of nucleotide bases on a single-stranded oligonucleotide by passing the oligonucleotide through a small hole of the order of 1 nanometer in diameter (a nanopore). Immersion of the nanopore in a conducting fluid and application of a potential across the nanopore results in a slight electrical current due to conduction of ions through the nanopore. The amount of current that flows through the nanopore is sensitive to the size of the nanopore. As an oligonucleotide passes through a nanopore, each nucleotide base obstructs the nanopore to a different degree. This results in a detectable change in the current passing through the nanopore allowing detection of the order of nucleotide bases in an oligonucleotide. See Branton, Daniel, et al. “The potential and challenges of nanopore sequencing.” Nanoscience and technology: A collection of reviews from Nature Journals (2010): 261-268. One example of a nanopore sequencer is the Oxford Nanopore MinION® sequencer.

The sequencer 112 may be connected to a computing device 114. The computing device 114 may be any type of conventional computing device such as a laptop computer, a desktop computer, a tablet, or the like. In some implementations, the sequencer 112 and the computing device 114 may be integrated into a single device. The sequencer 112 and the computing device 114 may be operated by a purchaser or potential purchaser of the item 102. Thus, by use of publicly available tag descriptions in the electronic record 108 and compact sequencers 112 such as nanopore sequences the techniques of this disclosure provide a way for users to independently determine the authenticity of the item 102.

The sequencer 112 together with the computing device 114 generate one or more electronic files representing the order of nucleotide bases in the synthetic polynucleotides 104. These sequences output from the sequencer 112 are referred to as retrieved sequences 116. The retrieved sequences 116 are provided to the computing device 110 communicatively connected to the electronic record 108. In some implementations, the computing device 114 connected to the sequencer 112 and the computing device 110 communicatively connected to the electronic record 108 are in communicative connection with each other via a network such as the Internet.

The computing device 110 may compare the retrieved sequences 116 to the original sequences 106 to determine if they have at least a threshold level of similarity. In many implementations, this will involve comparing thousands, millions, or more sequence strings each with hundreds of bases. This may require a significant number of computational operations to perform the comparison in a short amount of time such as less than five minutes, less than one minute, less than 30 seconds, or less than 10 seconds. Thus, utilizing cloud resources or network devices such as the electronic record 108 and the computing device 110 removes the computational burden from the computing device 114. This allows users with a computing device 114 with less processing power to promptly receive a determination of authenticity of the item 102. If there is a match, then an indication of authenticity 118 is returned from the computing device 110 to the computing device 114. The computing device 114 may then display a notification to a user that the item 102 is authentic. If the retrieved sequences 116 do not match the original sequences 106, the computing device 110 may return an indication that the item 102 is not authentic or that the validation failed.

If the item 102 is authentic, the synthetic polynucleotides 104 are the same polynucleotides placed on the item when it was initially tagged. However, damage to the synthetic polynucleotides 104 while placed on the item 102 and errors in sequencing may result in the retrieved sequences 116 being different from the original sequences 106. Moreover, the retrieved sequences 116 may represent the sequences of fewer than all of the synthetic polynucleotides 104 strands initially applied to the item 102. Thus, less than perfect identity in the two sets of sequences may still be considered a match if there is at least a threshold level of similarity. The threshold may be set as any value and may be adjusted for greater or lesser stringency. The level of stringency required may be based on the amount of damage likely sustained by synthetic polynucleotides 104 while on the item 102, the sequencing technique, and/or the value of the item 102.

For example, the threshold level may be at least 80% identity such as at least 85%, 90%, 95%, 96%, 97%, 98%, or 99% identity between the original sequences 106 and the retrieved sequences 116. The threshold level of similarity may also be based on the retrieved sequences 116 corresponding to at least a threshold number of the original sequences 106 (e.g., 10%, 20%, 30%, 40%, 50%, 60%, 70%, 80%, or 90%). For example, if 10 million synthetic polynucleotides 104 were originally placed on the item 102 and the retrieved sequences 116 contains one million sequences, a match may be identified if the one million retrieved sequences 116 are among the original sequences 106. Thus, a threshold level of similarity may include percent of sequence identity between individual strands in the original sequences 106 and the retrieved sequences 116 as well as recovering at least a threshold number of the original sequences 106.

The percent of sequence identity of two sequences may be determined by any one of a number of techniques used in bioinformatics or computer science and known to those of ordinary skill in the art. Examples include used in bioinformatics include software such as the BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). The Burrows-Wheeler Alignment tool (BWA) alignment tool may also be used to compare the similarity of sequences (Li H, Durbin R. Fast and accurate short read alignment with Burrows-Wheeler transform. Bioinformatics. 2009; 25(14): 1754-1760). Multiple algorithms for string comparison are discussed in D. Gusfield, Algorithms on Strings, Trees, & Sequences, New York, USA: Cambridge University Press, 1997.

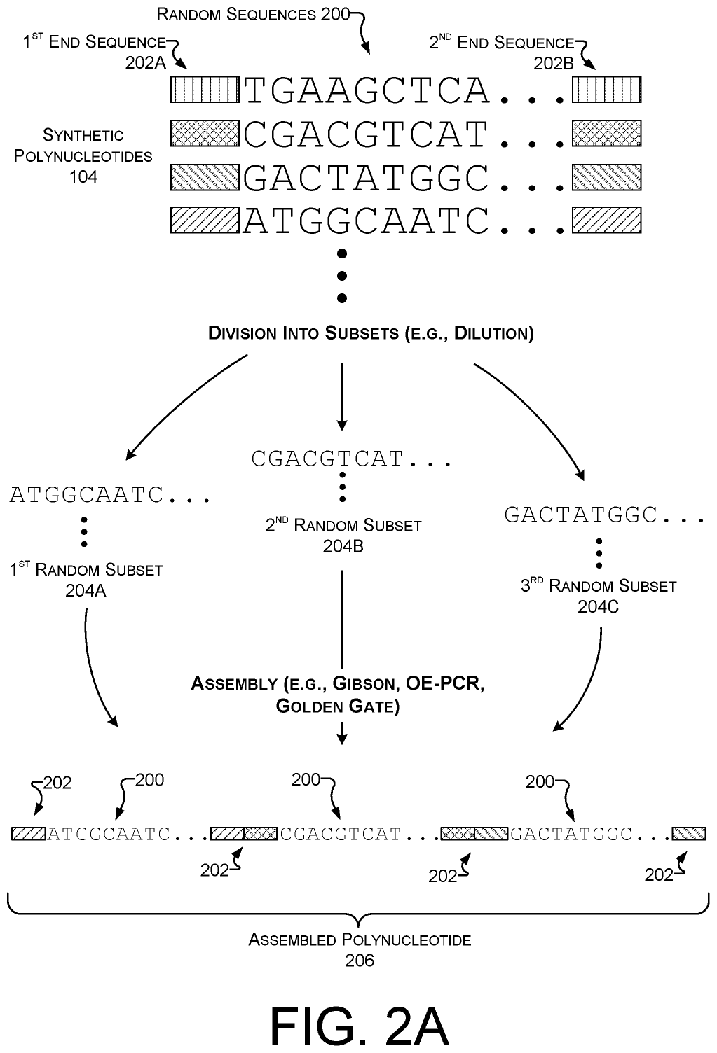

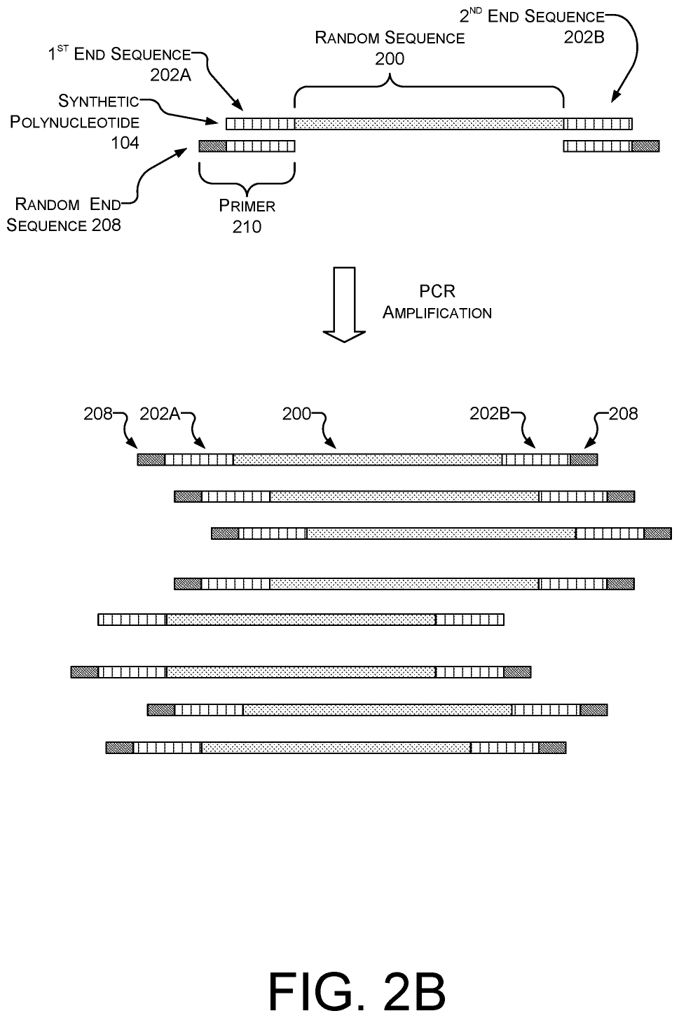

FIG. 2A shows additional details of the synthetic polynucleotides 104 and techniques for increasing the complexity of the anti-counterfeit tag 100. In some implementations, the entire length of the synthetic polynucleotides 104 are random sequences 200. However, in other implementations, each of the synthetic polynucleotides 104 includes a random sequence 200 and one or more sequences that are not random. The non-random sequences may be end sequences 202 that are present on one or both ends of the synthetic polynucleotides 104. Thus, the synthetic polynucleotides 104 may have a random sequence 200 in the middle flanked by a first end sequence 202A and a second end sequence 202B. The end sequences 202 may be synthesized by any one of multiple techniques known to persons of ordinary skill in the art for synthesizing polynucleotides with specific base-by-base sequences. Although illustrated in FIG. 2A as single-stranded molecules, the synthetic polynucleotides 104 may be double-stranded and one or both of the end sequences 202 may be sticky ends with overhangs.

The single-stranded polynucleotides could be amplified by standard PCR techniques as described above to create double-stranded polynucleotides. Blunt ends of the PCR amplification products may be converted to sticky ends by enzymatic digestion with a restriction enzyme that creates a staggered cut. The sticky end or overhang includes at least one nucleotide and may include many more such as about 5, 10, 15, or 20. Sticky ends can also be made without use of restriction enzymes such as described in Walker A., et al. A method for generating sticky-end PCR products which facilitates unidirectional cloning and the one-step assembly of complex DNA constructs. Plasmid. 59(3):155-62 (2008).

The end sequences 202 are illustrated as rectangles adjacent to the random sequences 200 in the synthetic polynucleotides 104. Each of the rectangles represents a string of nucleotide bases. The end sequences 202 may be any length and can be, for example, about 10-40 nucleotides long such as about 20 nucleotides long or about 30 nucleotides long. The first end sequence 202A and the second end sequence 202B may be the same length or different lengths.

A total length of the individual synthetic polynucleotides 104 may depend on the technique used to synthesize the polynucleotides. Phosphoramidite synthesis can synthesize polynucleotides accurately to a maximum length of about 300 nucleotides. See Palluk, S., Arlow, D. H., Rond, T., de, Barthel, S., Kang, J. S., et al. (2018). De novo DNA synthesis using polymerase-nucleotide conjugates. Nat. Biotechnol. 36, 645-650. Thus, the random sequences 200 may have a length of about 100-300 nucleotides, about 100 nucleotides, about 150 nucleotides, about 200 nucleotides, about 250 nucleotides, or about 300 nucleotides. Improvements in phosphoramidite synthesis technology may increase this maximum length above 300 nucleotides.

Enzymatic polynucleotides synthesis can create polynucleotides that are many thousands of nucleotides long. See Tang L, Tjong V, Li N, Yingling Y G, Chilkoti A, & Zauscher S (2014). Enzymatic polymerization of high molecular weight DNA amphiphiles that self-assemble into star-like micelles. Advanced Materials, 26(19), 3050-3054. Synthetic polynucleotides 104 synthesized by enzymatic synthesis may have a range of lengths due to variations in the number of polynucleotides incorporated at different strands by the enzymatic synthesis process. Thus, synthetic polynucleotides 104 synthesized by an enzymatic method may be described as having one average length although there will be variations in length for some of the individual polynucleotides. In some implementations, the average length of the synthetic polynucleotides 104 is greater than 400 nucleotides. For example, the average length of the synthetic polynucleotides 104 may be about 1000 nucleotides, about 5000 nucleotides, about 10,000 nucleotides, or another length greater than 400 nucleotides.

An end sequence 202 may be an artifact remaining from solid-phase synthesis such as a linker sequence or an artifact from enzymatic synthesis such as an initiator sequence. One or both of the end sequences 202 may be regions of the synthetic polynucleotides 104 that are used to assemble multiple polynucleotides together as discussed below. One or both of the end sequences 202 may be primer binding sites designed to hybridize with PCR primers. Primers may be designed to hybridize with end sequences 202 that are linker sequences or initiator sequences. Techniques for designing PCR primers and techniques for evaluating the suitability of primer sequences are well known to persons of ordinary skill in the art. For example, the first end sequence 202A may be a forward primer binding site and the second end sequence 202B may be a reverse primer binding site. In some implementations, all of the synthetic polynucleotides 104 may have the same forward and reverse primer binding sites. This makes it possible to use a single set of primers for PCR amplification of the entire set of synthetic polynucleotides 104.

As mentioned above, the population or collection of synthetic polynucleotides 104 with random sequences may include a very large number of individual polynucleotides such as millions or billions. This original set of synthetic polynucleotides 104 may be divided into two or more subsets that each contain a smaller number of polynucleotides. Because each polynucleotide in the original set of synthetic polynucleotides 104 will typically have a unique sequence, each of the subsets will thus include polynucleotides with sequences that are not found in any of the other subsets. The subsets may be random subsets 204 generated by taking a random selection of the synthetic polynucleotides 104.

One technique for generating one or more random subsets 204 from a collection of synthetic polynucleotides 104 in solution is to divide a sample into multiple portions such as by taking aliquots from a liquid sample. In an implementation, the liquid sample could be diluted by increasing its volume and then an aliquot of the diluted sample could be used as a random subset 204. For example, the 10 μL sample could be diluted tenfold by increasing its volume to 100 μL and an aliquot may be taken by removing 10 μL. The 10 μL aliquot would contain a random subset of about 10% of the polynucleotides that were present in the original sample. For example, if 10 million random polynucleotides were initially synthesized, the sequential 1:10 dilution and aliquoting described above would include a random selection of about a million of those polynucleotides. One batch of synthetic polynucleotides 104 may be synthesized and divided into multiple subsets that are each used to tag different items.

Other techniques for generating a random subset 204 include use of polynucleotide probes with random sequences anchored to magnetic beads. Although referred to as “random” subsets, it is not required that selection of the polynucleotides for includes in a subset is done in a manner that is strictly mathematically random. Ones of the synthetic polynucleotides 104 that happen to have energy-positive interactions with the probes can be selectively captured on the magnetic beads. The energy-positive interactions cause some sequences of polynucleotides to be preferentially bound to the random sequences on the magnetic beads. The binding may be, but is not limited to, hybridization between reverse complementary single-stranded polynucleotides. The magnetic beads may then be separated from the remainder of the polynucleotides and the attached polynucleotides eluted to create a random subset 204.

Alternatively, a random subset 204 may be created by PCR amplification with random primers that are, for example, about 5, 10, 15, or 20 nucleotides longs. This selectively amplifies those polynucleotides that have stronger interactions with the primers. It creates a population of polynucleotides in which the sequences that did not amplify are present at a much lower concentration and effectively removed from further processing because of the much higher concentration of the other polynucleotides that were amplified. Random primers, however, may hybridize to locations on the polynucleotides other than the ends creating multiple shorter sequences. Later matching would then use partial sequences rather than full-length sequences.

Multiple random subsets 204 may be generated from a collection of synthetic polynucleotides 104. FIG. 2A illustrates taking a first random subset 204A, a second random subset 204B, and a third random subset 204C of the synthetic polynucleotides 104. However, a greater or lesser number of random subsets 204 may be taken from the synthetic polynucleotides 104. In one implementation, the random subsets 204 may be generated by dividing a solution containing the synthetic polynucleotides 104 into multiple aliquots of equal volume. For example, if a solution containing the synthetic polynucleotides 104 was divided into three aliquots of equal volume, each aliquot would contain approximately one-third of the original number of synthetic polynucleotides 104.

One or more random subsets 204 may be taken from the synthetic polynucleotides 104 before sequencing. Doing so will decrease the complexity of the anti-counterfeit tag 100 by reducing the number of polynucleotides used to encode the anti-counterfeit tag 100, but it will also decrease the cost of sequencing necessary to characterize the anti-counterfeit tag 100. In some implementations, the number of synthetic polynucleotides included in a random subset 204 that is used as an anti-counterfeit tag 100 may be based on a value of the item 102. Less valuable items may be tagged with a random subset 204 that contains a fewer number of polynucleotides than more valuable items. For example, an item worth $1000 may be tagged with a 1:1000 subsample of the original set of synthetic polynucleotides 104 while an item worth $100,000 may be tagged with a 1:10 subsample.

In addition to being used to tune the number of polynucleotides included in an anti-counterfeit tag 100, random subsets 204 of the synthetic polynucleotides 104 may be used to generate pools of different nucleotides that may be assembled to create high-complexity polynucleotides. Due to the greater length and complexity these polynucleotides are more difficult for a bad actor to forge.

Longer polynucleotides with increased complexity are formed by assembling individual polynucleotides from two or more random subsets 204. Multiple techniques are known to persons of ordinary skill in the art for assembling polynucleotides such as Gibson assembly, Overlap-Extension Polymerase Chain Reaction (OE-PCR), and Golden Gate assembly.

Gibson assembly is an isothermal and single-reaction method for assembly of multiple DNA sequences described in Gibson, D., Young, L., Chuang, R Y. et al. Enzymatic assembly of DNA molecules up to several hundred kilobases. Nat Methods 6, 343-345 (2009). Gibson assembly is frequently used for the assembly of synthetic gene constructs in molecular cloning and synthetic biology due to its modularity and ease of use. A polynucleotide is amplified by using primers specific to its ends (e.g., end sequences 202A and 202B) and can be used as the starting material for a second assembly. It is possible to perform multiple rounds of Gibson assembly to join multiple polynucleotide strands together. Techniques for using Gibson assembly to assemble synthetic polynucleotides are described in Lopez, R., Chen, Y J., Dumas Ang, S. et al. DNA assembly for nanopore data storage readout. Nat Commun 10, 2933 (2019).

OE-PCR can be used as a simple approach to insert polynucleotide fragments into plasmids or to join different polynucleotide fragments together. OE-PCR is described in Anton V. Bryksin and Ichiro Matsumura. Overlap extension PCR cloning: a simple and reliable way to create recombinant plasmids. BioTechniques 48:6, 463-465 (2010). In the first step of PCR, overlapping sequences between each polynucleotide group can be created by using primers containing a 5′ overhang complementary to an overhang on the molecule it is joined to. For example, the first end sequence 202A of a polynucleotide from a first random subset 204A may be joined to the second end sequence 202B of a polypeptide from a second random subset 204B. All amplified random subsets 204 are mixed together, and polypeptides from random subsets with overlapping regions can be fused together via PCR with N cycles (N equals the number of random subsets). Finally, the outermost primers are used to selectively amplify the full length of multiple-fused polynucleotides. Techniques for using OE-PCR assembly to assemble synthetic polynucleotides are described in Lopez et al. (2019).

Golden Gate assembly is a molecular cloning method that allows simultaneous and directional assemble of multiple polynucleotides into a single strand using Type IIs restriction enzymes and T4 DNA ligase. Type IIs restriction enzymes cut DNA outside of their recognition sites and, therefore, can create non-palindromic overhangs. Because 256 potential overhang sequences are possible, multiple polynucleotides can be assembled by using combinations of overhang sequences. Techniques for performing Golden Gate assembly are described in Engler C, Kandzia R, Marillonnet S. A one pot, one step, precision cloning method with high throughput capability. PLoS ONE 3: e3647 (2008) and Engler C., Gruetzner R., Kandzia R., Marillonnet S. Golden Gate Shuffling: A One-Pot DNA Shuffling Method Based on Type IIs Restriction Enzymes. PLoS ONE 4(5): e5553 (2009).

Polynucleotides from multiple different random subsets 204 are assembled to create an assembled polynucleotide 206. The assembled polynucleotide 206 may be formed from two, three, or more different random subsets 204. An assembled polynucleotide 26 includes at least two random sequences 200 separated by two end sequences 202 that are not random. Assembly creates a pool of assembled polynucleotides 206 that each include a different combination of polynucleotides from the random subsets used to generate the assembled polynucleotides 206. This process induces an additional random element into the generation of the polynucleotides in an anti-counterfeit tag 100. In addition to the random sequences of nucleotides, there is also the random selection of individual polynucleotides from separate random subsets.

The individual polynucleotides from each random subset 204 that are joined together are themselves selected randomly from the respective subsets 204. This creates an additional level of complexity that cannot be replicated simply by repeating the same assembly process. Even if a bad actor could create the same random subsets of polynucleotides, individual polynucleotides would need to be isolated from the random subsets and separated into different reactions in order to create assembled polynucleotides 206 with the same sequences.

As described above, the end sequences 202 of synthetic polynucleotides 104 may include restriction sites, homologous overlapping regions, or other non-random sequences that function in a specific assembly technique to join polynucleotide strands together. In some implementations, synthetic polynucleotides 104 in each of the random subsets 204 have end sequences 202 that are different from the end sequences 202 in the other random subsets 204. For example, the polynucleotides in a first random subset 204A may include end sequences 202 that are different than the end sequences 202 of the polynucleotides in a second random subset 204B.

The variations in the end sequences 202 of the random subsets 204 may cause the polynucleotides from the various random subsets 204 to assemble in a specific order. For example, if an assembled polynucleotide 206 is created from a first random subset 204A, a second random subset 204B, and a third random subset 204C, the respective end sequences 202 on the individual polynucleotides in each of the random subsets 204 may specify the order in which the polynucleotides are assembled. For example, polynucleotides from the first random subset 204A may join to one end of the polynucleotides from the second random subset 204B and polynucleotides from the third random subset 204C may join to the other end of the polynucleotides the second random subset 204B. In this example, polynucleotides from the first random subset 204A are not able to join directly to polynucleotides from the third random subset 204C.

If overlapping sequences are used to join together polynucleotides for assembly, the second end sequence 202B of polynucleotides from the first random subset 204A may hybridize to the first end sequence 202A of polynucleotides in the second random subset 204B. The second end sequence 202B of polypeptides from the second random subset 204B may then hybridize to the first and sequence 202A of polynucleotides from the third random subset 204C. Thus, the order of joining polynucleotides from the various random subsets 204 may be controlled by the design of overlapping sequences used as the end sequences 202.

The assembled polynucleotides 206 can be sequenced after creation. Thus, the original sequences 106 shown in FIG. 1 may be the sequences of a large number of assembled polynucleotides 206. An anti-counterfeit tag 100 that comprises assembled polynucleotides 206 may use several thousands, millions, or billions of individual assembled polynucleotides 206. The assembled polynucleotides 206 can then be applied an item 102 as described above.

Assembly of multiple polynucleotides together may create assembled polynucleotides 206 that are longer than about 300 nucleotides, and thus, unable to be directly synthesized by phosphoramidite synthesis. To forge an anti-counterfeit tag 100 that uses assembled polynucleotides 206, a bad actor would either need to perform assembly of shorter polynucleotides created by phosphoramidite synthesis (which would be difficult or impossible to recreate the specific combinations achieved by random assembly) or use enzymatic synthesis to synthesize a polynucleotide with a specific sequence. Recreating the same pool of assembled polynucleotides 206 is difficult because the bad actor would need to perform many separate assembly reactions. Specifically, the bad actor would need to perform the same number of assembly reactions as the number of different assembled polynucleotides 206 which could be many millions or billions.

FIG. 2B shows one technique for placing additional random end sequences 208 on the ends of the end sequences 202. Thus, in some implementations, the end sequences 202 themselves may be flanked by an additional random sequence. The random end sequences 208 may be created when the synthetic polynucleotides 104 are synthesized by first generating random sequences followed by nonrandom sequences which in turn is then followed by generation of a longer a random sequence. Alternatively, the random end sequences 208 may be added to the synthetic polynucleotides 104 after synthesis by use of primers 210 with random overhangs. PCR amplification using primers 210 with random overhangs will generate double-stranded polynucleotides that are complementary to the random portions of the primers 210 as well as the nonrandom portions. Either technique, or a different technique, creates a population of synthetic polynucleotides 104 that include nonrandom sequences but also have random sequences at one or both ends. Thus, the end sequences 202A and 202B are positioned between two random sequences.

Synthetic polynucleotides 104 that have random end sequences 208 are more difficult for a bad actor to copy. If the very ends of the synthetic polynucleotides 104 are not random sequences, such as the first end sequence 202A and the second end sequence 202B as shown in FIG. 2A, a bad actor may be able to use PCR with primers that hybridized to the end sequences 202 to copy the synthetic polynucleotides 104. The bad actor may then use those copied molecules as fake tags on a forged or counterfeit item.

However, if there are random sequences on the very ends of the polynucleotides (i.e., the random end sequences 208) PCR amplification using primers hybridized to the end sequences 202 will not copy the entire length of the synthetic polynucleotides 104. The portions of the synthetic polynucleotides 104 that are not between the primer binding sites will not be copied. Validation of the retrieved sequences 116 by comparison to the original sequences 106 can identify the lack of the random end sequences 208. The validation may simply identify that there are no random end sequences 208. Or the validation may check the specific sequences of the random end sequences 208. To characterize the sequences of nucleotides in the random end sequences 208 there may need to be a sufficient number of molecules so that some can be sequenced and others applied to the item 102. For example, if there are total 5 bases in a random end sequence 208, there would be 4¹⁰ (for two end sequences)=1 million random sequences. If there are a billion molecules created by PCR, this would give ˜1000 copies of each sequence. A sufficient number to both sequence and apply to the item 102. However, as the length of the random end sequences 208 increases the number of copies of each sequence will decrease. With a long random end sequence 208 there may not be enough copies of the polynucleotides with each random end sequence 208 to both sequence and use for tagging the item 102. Thus, a length of the random end sequences 208 may be between about 2-7 nucleotides such as, for example, 2, 3, 4, 5, 6, or 7 nucleotides long. In order to make full-length copies of the synthetic polynucleotides 104, unique primers would be needed for each of the synthetic polynucleotides 104. The bad actor would need to create a large number (e.g., millions or more) of separate primers making it difficult and costly to copy existing polynucleotides.

An alternative technique to prevent a bad actor from using primers to PCR amplify and copy an anti-counterfeit tag 100 is to remove or truncate the end sequences 202A and 202B so that they can no longer function as primer binding sites. There are many techniques known to those of ordinary skill in the art for cleaving the ends of polynucleotides that have known sequences. This can be done, for example, by enzymatic digestion such as USER digest, restriction enzyme digestion, RNA digestion, UV cut, or other technique.

For example, the primers may include deoxy-uracil to introduce uracil bases at the junction of the end sequence 202 to the random sequence 200. The USER digest breaks the phosphodiester backbone of a polynucleotide by using a uracil cleavage system in which the sequential addition of Uracil DNA Glycosylase (UDG) and endonuclease VIII generates a single nucleotide gap at the location of a uracil base in polynucleotide containing a deoxy-uracil. UDG catalyzes the excision of the uracil base, creating an abasic site with an intact phosphodiester backbone. The lyase activity of Endonuclease VII breaks the phosphodiester backbone both 3′ and 5′ to the abasic site, liberating the deoxyribose sugar.

As a further example, the end sequences 202 may be designed with sequences that are recognized and cleaved by a restriction endonuclease. If the end sequences 202 are not fully removed, they may be truncated to create a truncated end sequence. The truncated end sequence are too short (e.g., 1-5 nucleosides) to function as a primer binding site.

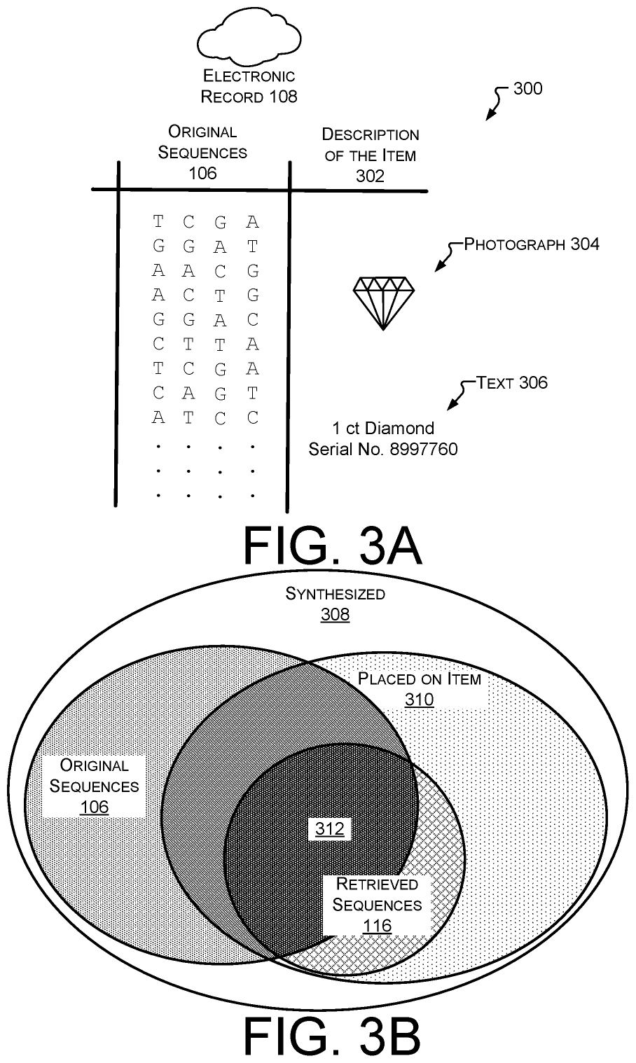

FIG. 3A shows an entry 300 in electronic record 108. As described above, the electronic record 108 may be maintained on one or more network-accessible computing devices at one or more locations physically distant from the item (i.e., a cloud-based system). Each entry 300 in electronic record 108 includes the original sequences 106 and description of the item 302. Electronic record 108 may include entries for multiple different items. In some implementations, electronic record 108 may be implemented as a list, a table, an array, a spreadsheet, a database, or another data structure.

The original sequences 106 may be in any electronic format used for storing representations of nucleotides such as ASCII or FASTA. Although only four partial sequences are shown in FIG. 3, the original sequences 106 will in most implementations include a much larger number of unique sequences of greater length such that manipulation other than by a computer would be impractical or impossible.

The description of the item 302 may include, for example, a photograph 304 and/or the text description 306 of the item. Other types of descriptions of the item 302 are also possible such as, for example, a description of another taggant placed on the item such as a serial number or code. Description of the item 302 is used to identify the item 102 tagged with the synthetic polynucleotides 104.

Once the original sequences 106 of the synthetic polynucleotides 104 are known, the original sequences 106 and the description of the item 302 can be registered in the electronic record 108. Entry 300 may be registered in the electronic record 108 by uploading the original sequences 106 from the sequence computing device used to generate the sequences and by uploading a description of the item 302. A description of the item 302 may be uploaded from a different computing device than the original sequences 106. The original sequences 106 and the description of the item 302 may be uniquely linked, associated, joined, or correlated in the electronic record 108 with each other.

The entry 300 may also include a description of where the synthetic polynucleotides 104 are located on the item 102. For example, the entry 300 may describe where on the outside surface of the item 102 the synthetic polynucleotides 104 were placed. If the item 102 is liquid, the entry 300 may indicate that the synthetic polynucleotides 104 are included in the liquid rather than on a container. This can guide collection of the polynucleotides for the purpose of validating the authenticity of the item 102.

FIG. 3B is a Venn diagram illustrating the relationship between different sets of polynucleotide strands and polynucleotide sequences. The largest circle 308 represents the synthetic polynucleotide strands with random sequences. This is the totality of all the molecules created when a batch of synthetic polynucleotides is synthesized. The synthetic polynucleotides 104 may be created by a technique such as column synthesis or array synthesis that generates in one batch a very large number of unique polynucleotide strands such as 10⁶, 10⁸, 10¹², 10¹⁸, or 10²⁴ each with different, random sequences.

Some or all of the polynucleotide strands that were synthesized 308 are sequenced to generate the original sequences 106. In some implementations, all of the polynucleotides that were synthesized are sequenced in which case circles 308 and 106 will be the same. But in other implementations, fewer than all of the synthesized polynucleotides 308 are sequenced either intentionally or unintentionally. Thus, the original sequences 106 may be sequences of only a portion of the polynucleotides that were synthesized 308. The size of the subset of the synthesized polynucleotides that are sequenced (i.e., circle 106) may be determined based on the value of the item.

The polynucleotides placed on the item 310 include polynucleotide strands that were sequenced as part of the original sequences 106 and may also include polynucleotide strands that were not sequenced. Thus, some of the polynucleotides 308 that were synthesized may be placed on the item without being sequenced. The overlap between circles 106 and 310 represents those polynucleotide strands for which the sequences are known and that are placed on the item.

The polynucleotides collected from the item and then sequenced generate the retrieved sequences 116. The retrieved sequences 116 include sequences of polynucleotides that were included in the original sequences 106 as shown by overlap area 312. The retrieved sequences 116 may also include sequences of polynucleotides placed on the item but not previously sequenced. The number of the retrieved sequences 116 relative to the polynucleotides places on the item 310 may be changed based on the value of the item.

Comparison of the original sequences 106 and the retrieved sequences 116 for the purpose of determining if the item is authentic is done with the sequences from overlap area 312. Sequences in this area 312 of the Venn diagram are both included among the original sequences 106 in the electronic record 108 and included in the retrieved sequences 116 recovered from the item. If there is sufficient similarity between these two subsets of sequences in terms of number of unique sequences and similarity between the sequences then the retrieved sequences 116 may be determined to “match” the original sequences 106 and the item may be deemed authentic.

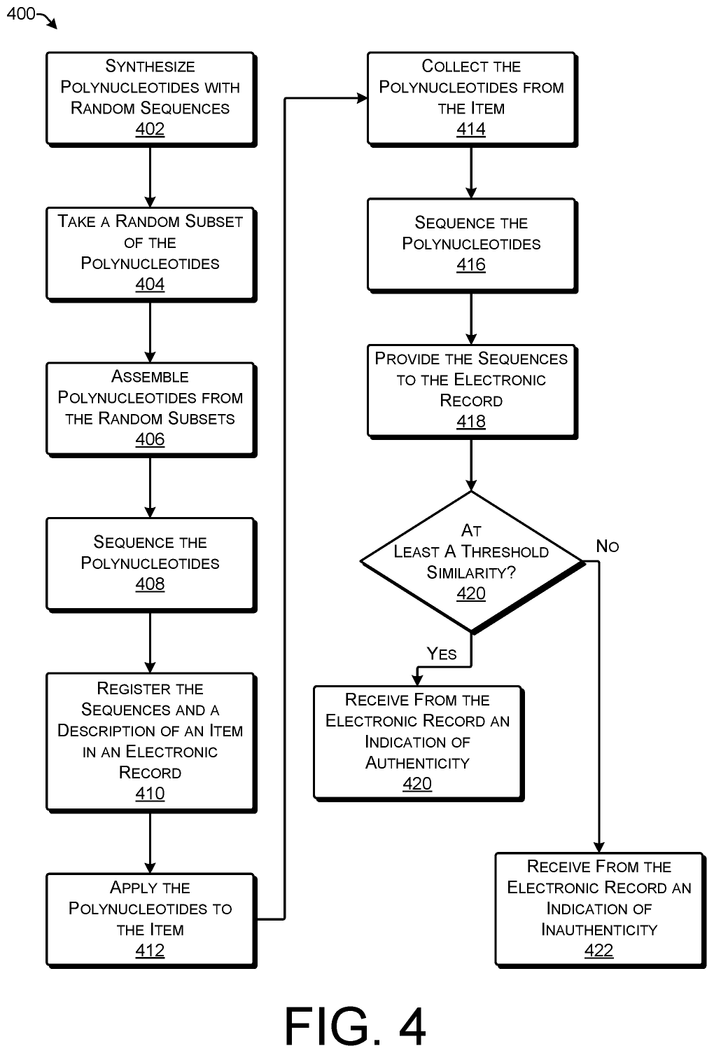

FIG. 4 shows an illustrative process 400 for tagging an item with an anti-counterfeit tag made from a plurality of polynucleotides having random sequences.

At operation 402, a plurality of synthetic polynucleotides comprising random sequences is synthesized. The polynucleotides may be synthesized by any technique that creates DNA or RNA strands such that at least a portion of the strands have a random sequence of nucleotide bases. Techniques are known to those of ordinary skill in the art for synthesizing polynucleotides with random sequences and include phosphoramidite synthesis and enzymatic synthesis. Synthesis will generally create one copy of each polynucleotide with a unique random sequence.

Polynucleotides with random sequences have sequences that are not specified in advance and have an order of nucleotide bases that is random or approximately random. Random sequences may be created by providing the sequencing system with multiple different nucleotides without specifying or limiting which base is incorporated. The next base incorporated in any given strand during a round of synthesis will be determined stochastically leading to the generation of random sequences.

With some synthesis techniques, random sequences may include approximately equal ratios of all nucleotide bases used for synthesizing the polynucleotides. Thus, synthetic polynucleotides with random sequences may be created by providing a mixture of nucleotide bases in approximately equal proportion. However, random sequences may also be generated in which the ratio of nucleotide bases is not equal. For example, a random nucleotides sequence may be created that has 30% G, 30% C, 20% A, and 20% T. Thus, a random sequence may include equal or unequal proportions of all the incorporated bases and may be formed by a technique that has a bias for incorporating one or more bases relative to the other bases. One technique for creating an anti-counterfeit tag using specific ratios of nucleotide bases is Counterfeit Tags Using Base Ratios of Polynucleotides” and filed the same day as this application.

The plurality of synthetic polynucleotides may include a large number of polynucleotides such as many thousands, tens of thousands, hundreds of thousands, millions, or billions of different polynucleotides with unique, random sequences. A length of each of the synthetic polynucleotides may be between approximately 50 nucleotides and approximately 10,000 nucleotides. In some implementations, the synthetic polynucleotides may be synthesized by phosphoramidite synthesis, and a length of the synthetic polynucleotides may be about 100-300 nucleotides. In some implementations, the synthetic polynucleotides may be synthesized by enzymatic synthesis, and an average length of the synthetic polynucleotides may be greater than 400 nucleotides such as between about 400 and 10,000 nucleotides. Sequences with lengths shorter than 400 nucleotides may also be synthesized by enzymatic synthesis.

One or more portions of the synthetic polynucleotides may include non-random sequences. Non-random sequences may be located at one or both ends (e.g., 3′ end and/or 5′ end) of the synthetic polynucleotides. Non-random sequences located on an end of the synthetic polynucleotides may be referred to as end sequences such as those illustrated in FIG. 2A. If non-random end sequences are included the synthetic polynucleotides may also contain additional random sequences outside of the end sequences as shown in FIG. 2B.

The non-random sequences may be sequences that have a role in the synthesis of the polynucleotides. For example, the non-random sequences may be linker sequences used to attach the polynucleotides to a solid substrate for solid-phase synthesis. As a further example, the non-random sequences may be initiator sequences used by an enzyme such as TDT to initiate enzymatic synthesis and extension of the polynucleotides strand.

The non-random sequences may alternatively or additionally have a role in later processing of the polynucleotides. For example, the non-random sequences may be primer binding sites. The primer sites may be used for PCR amplification of the polynucleotides. In an implementation, each of the synthetic polynucleotides may include a forward primer binding sites and reverse primer binding site that are not random. Further, each of the synthetic polynucleotides may have the same forward primer binding site and reverse primer binding site so that all of the polynucleotides can be amplified with the same pair of primers. Design and use of polynucleotides primers are well known to persons of ordinary skill in the art. A length of the primer binding sites may be about 10-30 nucleotides and the non-random sequences may be designed using software and conventional techniques. Techniques for primer design are known to those of ordinary skill in the art.

At operation 404, a random subset of the plurality of synthetic polynucleotides generated at operation 402 may be taken for use as the anti-counterfeit tag. The random subset may be taken by dividing a sample containing the synthetic polynucleotides. For example, a sample of the polynucleotides may be divided into a first random subset and a second random subset by first diluting the synthetic nucleotides and then splitting the diluted polynucleotides into two equal volume portions. Other techniques for taking a random subset of the polynucleotides are also possible. More than two random subsets may also be created.

Taking a random subset of the plurality of synthetic polynucleotides is optional. If a random subset is not taken, all or substantially all of the synthetic polynucleotides generated at operation 402 may be used as the anti-counterfeit tag.

Taking a random subset from the synthesized polynucleotides produces a smaller number of polynucleotides that can be used for the anti-counterfeit tag. Thus, in some implementations, synthetic polynucleotides with random sequences may be synthesized in excess and only a portion of those synthetic polynucleotides are used to tag a specific item. Also, the synthetic polynucleotides synthesized at operation 402 may be divided into multiple random subsets and used to tag multiple different items. The cost of forging an anti-counterfeit tag depends on the length and number of polynucleotides that must be synthesized to reproduce the tag. There is less incentive to forge a counterfeit tag for lower value items than for higher value items. Accordingly, the number of synthetic polynucleotides in one or more random subsets used to tag an item may be based on the value of the item (i.e., more polynucleotides can be used to tag more expensive items).

At operation 406, randomly selected synthetic polynucleotides from two or more of the random subsets generated at operation 404 are assembled to generate a plurality of assembled polynucleotides. For example, randomly selected synthetic polynucleotides from the first random subset 204A and the second random subset 204B shown in FIG. 2A may be assembled to generate the plurality of assembled polynucleotides. The synthetic polynucleotides may be assembled using any one of multiple techniques for assembling polynucleotides known to those of ordinary skill in the art such as Gibson assembly, OE-PCR, or Golden Gate assembly.

Assembling the synthetic polynucleotides into longer assembled polynucleotides is an optional step that may be omitted. If assembly is not performed, the anti-counterfeit tag will comprise the synthetic polynucleotides in one of the random subsets as they were synthesized with any end sequences that may be present.

In an implementation, the assembled polynucleotides are assembled from three or more random subsets of the synthetic polynucleotides such as the first random subset 204A, the second random subset 204B, and the third random subset 204C shown in FIG. 2A. Assembly makes use of non-random end sequences on the synthetic polynucleotides to join the multiple polynucleotide strands together. The non-random end sequences for polynucleotides in any one of the random subsets are different from the end sequences in each of the other random subsets. Because the specific end sequences function in the assembly, an order of the assembly of the individual synthetic polynucleotides from the three or more random subsets is specified by the end sequences.

Joining multiple synthetic polynucleotides together creates longer polynucleotides with at least two random sequences separated by two end sequences (i.e., one end sequence from each of the two polynucleotides that are joined) that are not random. Using assembled polynucleotides that link together multiple random sequences increases the complexity of the anti-counterfeit tag. Assembling two synthetic polynucleotides together creates a lower complexity tag than assembling ten synthetic polynucleotides. Thus, another variable that can be tuned to adjust the complexity of an anti-counterfeit tag is the number of random subsets used for the creation of assembled polynucleotides. That number may be based on the value of the item. For example, a more valuable item can be tagged with an anti-counterfeit tag made from a greater number of random subsets than a less valuable item.

At operation 408, at least a portion of the synthetic polynucleotides are sequenced to obtain a plurality of original sequences. All or fewer than all of the synthetic polynucleotides synthesized at operation 402 may be sequenced. For example, a large number of synthetic polynucleotides with random sequences may be synthesized by column synthesis and only a fraction of those may be sequenced (e.g., 10¹⁸ unique random sequences synthesized and 10⁶ sequenced). Multiple techniques and devices for sequencing polynucleotides are known to those of ordinary skill in the art including sequencing-by-synthesis and nanopore sequencing. The plurality of original sequences are representations of the nucleotide bases in the synthetic polynucleotides as detected by a sequencer. Sequencers are known to generate errors the type and frequency of which vary by type of sequencer and operational parameters. Thus, the plurality of original sequences may not perfectly represent the order of nucleotide bases in the synthetic polynucleotides.

If one or more random subsets of the synthetic polynucleotides is taken at operation 404, the nucleotides in the one or more random subsets may be sequenced without sequencing the remainder of the synthetic polynucleotides generated at operation 402. Alternatively, only a portion of the entire batch of synthetic polynucleotides may be sequenced without taking a random subset. Thus, the undifferentiated set of synthetic polynucleotides may contain some nucleotide strands that are sequenced and some that are not.

If assembled polynucleotides are created at operation 406, the plurality of assembled polynucleotides is sequenced at operation 408. Thus, sequencing at least a portion of the synthetic polynucleotides includes sequencing the assembled polynucleotides following the assembly process.

Prior to sequencing, in some implementations, copies may be made of the synthetic polynucleotides so that there are multiple polynucleotide strands with each unique, random sequence. Thus, some polynucleotide strands can be sequenced and discarded while others are used to tag an item. Multiple copies of the polynucleotide strands may be made by anyone at multiple techniques known in the art such as PCR, other enzymatic techniques, and non-enzymatic techniques for creating multiple copies of existing polynucleotides.

PCR refers to a reaction for the in vitro amplification of specific DNA sequences by the simultaneous primer extension of complementary strands of DNA. In other words, PCR is a reaction for making multiple copies or replicates of a target nucleic acid flanked by primer binding sites. The reaction comprising one or more repetitions of the following steps: (i) denaturing the target nucleic acid, (ii) annealing primers to the primer binding sites, and (iii) extending the primers by a template-dependent polymerase in the presence of nucleoside triphosphates. Usually, the reaction is cycled through different temperatures optimized for each step in a thermocycler. A thermocycler (also known as a thermal cycler, PCR machine, or DNA amplifier) can be implemented with a thermal block that has holes where tubes holding an amplification reaction mixture can be inserted. Other implementations can use a microfluidic chip in which the amplification reaction mixture moves via a channel through hot and cold zones.

Each cycle doubles the number of copies of the specific DNA sequence being amplified. This results in an exponential increase in copy number. Particular temperatures, durations at each step, and rates of change between steps depend on many factors well-known to those of ordinary skill in the art, e.g., exemplified by the references: McPherson et al., editors, PCR: A Practical Approach and PCR 2: A Practical Approach (IRL Press, Oxford, 1991 and 1995, respectively). Illustrative methods for detecting a PCR product using an oligonucleotide probe capable of hybridizing with the target sequence or amplicon are described in Mullis, U.S. Pat. Nos. 4,683,195 and 4,683,202; EP No. 237,362.

However, it is also possible in some implementations to recover the synthetic polynucleotides following sequencing. Thus, make additional copies would not be necessary and the same molecules that are sequenced will later be applied to an item as the anti-counterfeit tag. Following sequencing, synthetic polynucleotides that are recovered and may be prepared for application to the item such as by cleaning or mixing with one or more stabilizing reagents.

At operation 410, the original sequences and a description of the item are registered in an electronic record. The registration may consist of creating an entry in the electronic record that links or otherwise associates the original sequences with the description of the item. The electronic record may also indicate where the synthetic polynucleotides are placed on the item. The electronic record may be a database, spreadsheet, table, list, or other data structure configured to store the original sequences and the description of the item. The electronic record may be maintained on a network-accessible computing device that is physically distant from the item and any devices used to synthesize or sequence the polynucleotides. In an implementation, the electronic record may be maintained in a cloud-based system.

The electronic record may be publicly available so that the original sequences and description of the item may be accessed by anyone. This enables any user with access to the electronic record, and the ability to sequence polynucleotides, to validate the authenticity of the item. Doing so removes reliance on assertions of authenticity provided by an expert or other third party.

However, in other implementations, access to the electronic record may be limited by any technique used to control access to an online database or electronic file. For example, a username and password may be required to access the original sequences in the electronic record. This provides an additional level of security by making it more difficult for a bad actor to identify which polynucleotides need to be synthesized to forge the anti-counterfeit tag.

At operation 412, the plurality of synthetic polynucleotides are applied to the item. If a random subset of the synthetic polynucleotides is taken at operation 404, the polynucleotides in that random subset are applied to the item. If assembled polynucleotides are created at operation 406, the plurality of assembled polynucleotides are applied to the item. Unlike other techniques for using polynucleotides as taggants that label an item with only a single polynucleotide sequence, the techniques of this disclosure use a large number of polynucleotides with different, random sequences that collectively function as the anti-counterfeit tag. The large number of polynucleotides may be at least 10¹⁰, at least 10¹², at least 10¹⁸, or more. If every polynucleotide synthesized is not sequenced at operation 408 but the entire batch of synthetic polynucleotides is applied to the item, then the synthetic polynucleotides applied to the item may include polynucleotides that were never sequenced.

The synthetic polynucleotides may be applied to the item in any number of different ways. The synthetic polynucleotides may be applied to the outside of the item or to packaging containing the item. If the item is liquid or powder, the synthetic polynucleotides may be mixed in with the item. In some implementations, the synthetic polynucleotides may be placed on, in, or under a visible taggant such as a QR code or holographic sticker. The synthetic polynucleotides applied to the item may be protected by a coating or encapsulating layer that can be applied together with the polynucleotides or after the polynucleotides have been applied to the item.

At operation 414, the plurality of synthetic polynucleotides are collected from the item. The synthetic polynucleotides may be collected using any established techniques for collecting polynucleotides from environmental or forensic samples. Following collection, the synthetic polynucleotides may be cleaned or processed in preparation for sequencing using commercial kits or any one of a number of techniques known to those of ordinary skill in the art.

If the item is authentic, then the polynucleotides collected from the item will be the same as the synthetic polynucleotides applied to the item at operation 412. If the item is a counterfeit or a forgery without an anti-counterfeit tag, there will be no polynucleotides to collect from the item. If the anti-counterfeit tag itself is not successfully forged, the polynucleotides collected from the item will have different sequences than the polynucleotides applied to the item and can be detected as such.

At operation 416, at least a portion of the plurality of the synthetic polynucleotides collected from the item are sequenced. The polynucleotides collected from the item may be sequenced using any sequencing technology such as, for example, nanopore sequencing. The method of sequencing used at operation 416 may be the same or different than the method of sequencing used at operation 408.

The portion of the plurality of synthetic polynucleotides that is sequenced includes more than one polynucleotide strand and may include at least 10⁴ polynucleotides, at least 10⁸ polynucleotides, at least 10¹² polynucleotides, or at least 10¹⁸ polynucleotides. In some implementations, fewer than all of the polynucleotides collected from the item at operation 414 are sequenced. In some implementations, polynucleotides that were not initially sequenced at operation 408 may be sequenced. For example, if fewer than all the synthetic polynucleotides are sequenced at operation 408, some of those polynucleotide strands that were not initially sequenced but were applied to the item may be sequenced at operation 416.

It may be possible to validate the authenticity of the item by sequencing and evaluating only a portion of the polynucleotides that are applied to the item. Sequencing fewer than all of the synthetic polynucleotides collected reduces the sequencing cost, and thus, reduces the cost to validate the authenticity of the item. The size of the portion of the synthetic polynucleotides collected from the item that is sequenced may be based on the desired level of confidence in the accuracy of the validation. Lower levels of confidence in the accuracy of the validation may be acceptable for lower value items. Thus, the size of the portion of the polynucleotides that is sequenced may be based on a value of the item. The larger portion of the polynucleotides may be sequenced for higher value items and a smaller portion of the polynucleotides may be sequenced for lower value items.

The output generated by sequencing the polynucleotides collected from the item is a plurality of retrieved sequences. The plurality of retrieved sequences represents the order of nucleotide bases in the polynucleotides collected from the item as detected by the sequencing system. The plurality of retrieved sequences may be represented electronically in a computer file.

At operation 418, the plurality of the retrieved sequences are provided to a computing device communicatively connected to the electronic record. In some implementations, a computer file containing the plurality of retrieved sequences may be transmitted over a communications network such as the Internet from a computing device coupled to the sequencer to a network-based computing device that stores or maintains the electronic record.

At operation 420, it is determined if the item is authentic by determining that the retrieved sequences obtained at operation 416 have at least a threshold level of similarity to sequences included in the original sequences obtained at operation 408. Comparison of the plurality of the retrieved sequences to the plurality of original sequences to determine if there is a 100% match or a partial match between retrieved sequences and some of the original sequences. Even for authentic items in which synthetic polynucleotides in the anti-counterfeit tag have not changed there may be differences in the retrieved sequences obtained when validating the item as compared to the original sequences obtained when the polynucleotides were first placed on the item. The differences may arise from errors in sequencing either initially or at the time of validation. The differences may also arise from damage that occurs to the polynucleotides.

Accordingly, comparing the two sets of sequences may determine that there is a “match” so long as there is at least a threshold level of similarity even if there is not perfect identity between the two sets of sequences. The threshold level of similarity may be any threshold such as, for example, about 80% similarity or higher. If fewer than all of the synthetic polynucleotides applied to the item are sequenced (to reduce sequencing costs), the item may be identified as authentic if those polynucleotides that are sequenced are found in the plurality of original sequences even though there is no match for all of the original sequences. For example, if the item is labeled with 1,000,000 polynucleotides and only 100,000 are sequenced at operation 416, a determination that those 100,000 sequences are found among the original set of sequences may be sufficient to identify the item as authentic.

There may also be a “match” if some of the retrieved sequences do not match any of the original sequences. This can occur in items that are authentic if only a portion of the synthetic polynucleotides applied to the item is initially sequenced at operation 408. There may be some synthetic polynucleotides that are not represented in the original sequencing at operation 408 but are among the polynucleotides collected from the item and sequenced. Thus, the retrieved sequences may be determined to represent the same set of polynucleotides (indicating the item is authentic) if at least a threshold number of the retrieved sequences are found among the original sequences. There may be sequences in the retrieved sequences that are not found in the original sequences and/or sequences in the original sequences that are not found in the retrieved sequences. In practice there will likely be both.

Thus, a threshold level of similarity between the set of polynucleotide sequences represented by the original sequences and the set of polynucleotide sequences represented by the retrieved sequences may be determined by identifying at least a threshold number of sequences that are the same or similar between the two sets of sequences. For example, the threshold level of similarity may be at least 10⁶, 10¹⁰, 10¹⁴, or 10¹⁸ sequences from the retrieved sequences having at least a threshold level of similarity (e.g., 95% identity) to sequences in the set of original sequences.

The determination of similarity between the two sets of sequences may be made by the computing device that maintains the electronic record. Thus, the comparison may be done by a computing device that is located in the cloud and managed by a third party. The third party may be an entity that is not otherwise associated with the item or the transaction of the item. Comparison of partial similarity between many millions or billions of sequenced strings may be a very computationally intensive operation that is difficult for conventional desktop computers or laptop computers to complete in a reasonable time. Cloud-based computing resources may be used to make such a comparison in a relatively short amount of time such as less than five minutes, less than one minute, less than 30 seconds, or less than 10 seconds.

If there is at least a threshold level of similarity between recovered sequences and at least a portion of the original sequences, process 400 proceeds along the “yes” path to operation 420. At operation 420, the computing device that is communicatively connected to the electronic record may generate an indication of authenticity and send that indication to a different computing device. The indication of authenticity may be displayed on the receiving computing device. The indication of authenticity may be an email or other electronic communication. In some implementations, the indication of authenticity may be encrypted. The computing device that receives the indication of authenticity may be a computing device used for sequencing the polynucleotides collected from the item or a different computing device such as another computing device under the control of a purchaser or potential purchaser of the item.

If, however, there is no match between the retrieved sequences and the original sequences or if the match has less than a threshold level of similarity then the item may be determined to be inauthentic. In which case process 400 proceeds along the “no” path to operation 422.

At operation 422, an indication of inauthenticity is received from the computing device that is communicatively connected to the electronic record. The indication of inauthenticity may be communicated to a receiving computing device that the item could not be authenticated and may be a counterfeit or a forgery. The receiving computing device may display an indication that the item could not be validated as authentic.

### Illustrative Computer Architecture

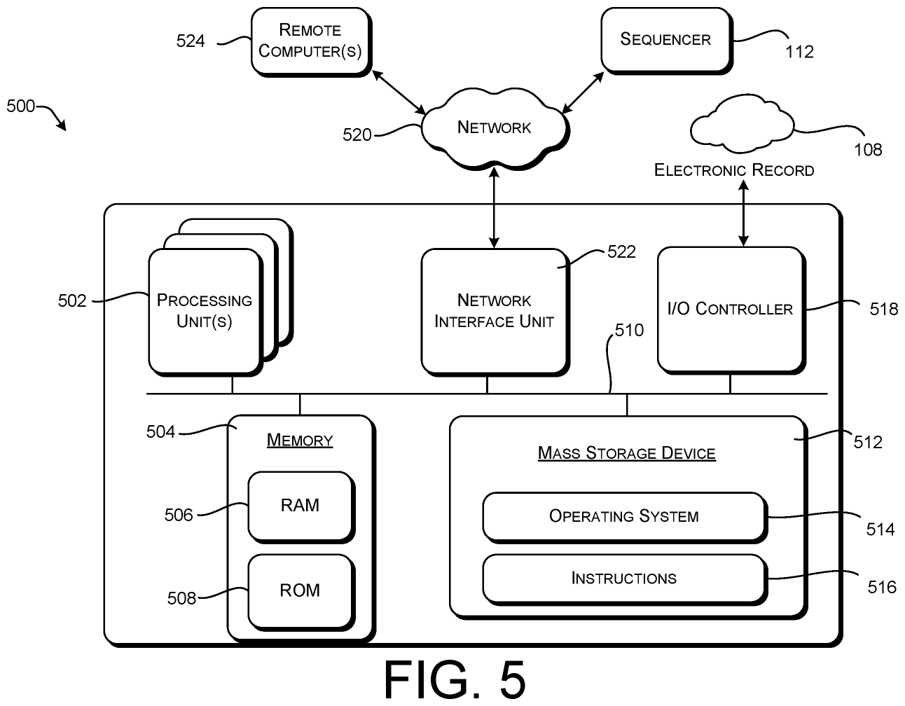

FIG. 5 is a computer architecture diagram showing an illustrative computer hardware and software architecture for a computing device such as the computing device 110 or the computing device 114 introduced FIG. 1. In particular, the computer 500 illustrated in FIG. 5 can be utilized to receive raw data from a sequencer 112 or to maintain the electronic record 108.

The computer 500 includes one or more processing units 502, a system memory 504, including a random-access memory 506 (“RAM”) and a read-only memory (“ROM”) 508, and a system bus 510 that couples the memory 504 to the processing unit(s) 502. A basic input/output system (“BIOS” or “firmware”) containing the basic routines that help to transfer information between elements within the computer 500, such as during startup, can be stored in the ROM 508. The computer 500 further includes a mass storage device 512 for storing an operating system 514 and other instructions 516 that represent application programs and/or other types of programs. The other programs may be, for example, instructions to compare retrieved sequences 116 to original sequences 106 and determine if there is at least a threshold level of similarity. The mass storage device 512 can also be configured to store files, documents, and data. In some implementations, electronic record 108 may be maintained in the mass storage device 512.

The mass storage device 512 is connected to the processing unit(s) 502 through a mass storage controller (not shown) connected to the bus 510. The mass storage device 512 and its associated computer-readable media provide non-volatile storage for the computer 500. Although the description of computer-readable media contained herein refers to a mass storage device, such as a hard disk, CD-ROM drive, DVD-ROM drive, or USB storage key, it should be appreciated by those skilled in the art that computer-readable media can be any available computer-readable storage media or communication media that can be accessed by the computer 500.

Communication media includes computer-readable instructions, data structures, program modules, or other data in a modulated data signal such as a carrier wave or other transport mechanism and includes any delivery media. The term “modulated data signal” means a signal that has one or more of its characteristics changed or set in a manner so as to encode information in the signal. By way of example, and not limitation, communication media includes wired media such as a wired network or direct-wired connection, and wireless media such as acoustic, radio frequency, infrared, and other wireless media. Combinations of any of the above should also be included within the scope of computer-readable media.

By way of example, and not limitation, computer-readable storage media can include volatile and non-volatile, removable and non-removable media implemented in any method or technology for storage of information such as computer-readable instructions, data structures, program modules, or other data. For example, computer-readable storage media includes, but is not limited to, RAM 506, ROM 508, EPROM, EEPROM, flash memory or other solid-state memory technology, CD-ROM, digital versatile disks (“DVD”), HD-DVD, BLU-RAY, 4K Ultra BLU-RAY, or other optical storage, magnetic cassettes, magnetic tape, magnetic disk storage or other magnetic storage devices, or any other medium that can be used to store the desired information and which can be accessed by the computer 500. For purposes of the claims, the phrase “computer-readable storage medium,” and variations thereof, does not include waves or signals per se or communication media.

According to various configurations, the computer 500 can operate in a networked environment using logical connections to a remote computer(s) 524 through a network 520. For example, if the computer 500 corresponds to computing device 114 then the remote computer 524 may correspond to the computing device 110. The computer 500 can connect to the network 520 through a network interface unit 522 connected to the bus 510. It should be appreciated that the network interface unit 522 can also be utilized to connect to other types of networks and remote computer systems. The computer 500 can also include an input/output controller 518 for receiving and processing input from a number of other devices, including a keyboard, mouse, touch input, an electronic stylus (not shown), or equipment such as a sequencer 112 for detecting the sequence of polynucleotides. Similarly, the input/output controller 518 can provide output to a display screen or other type of output device (not shown).

It should be appreciated that the software components described herein, when loaded into the processing unit(s) 502 and executed, can transform the processing unit(s) 502 and the overall computer 500 from a general-purpose computing device into a special-purpose computing device customized to facilitate the functionality presented herein. The processing unit(s) 502 can be constructed from any number of transistors or other discrete circuit elements, which can individually or collectively assume any number of states. More specifically, the processing unit(s) 502 can operate as a finite-state machine, in response to executable instructions contained within the software modules disclosed herein. These computer-executable instructions can transform the processing unit(s) 502 by specifying how the processing unit(s) 502 transitions between states, thereby transforming the transistors or other discrete hardware elements constituting the processing unit(s) 502.

Encoding software modules can also transform the physical structure of the computer-readable media presented herein. The specific transformation of physical structure depends on various factors, in different implementations of this description. Examples of such factors include, but are not limited to, the technology used to implement the computer-readable media, whether the computer-readable media is characterized as primary or secondary storage, and the like. For example, if the computer-readable media is implemented as semiconductor-based memory, the software disclosed herein can be encoded on the computer-readable media by transforming the physical state of the semiconductor memory. For instance, the software can transform the state of transistors, capacitors, or other discrete circuit elements constituting the semiconductor memory. The software can also transform the physical state of such components to store data thereupon.

As another example, the computer-readable media disclosed herein can be implemented using magnetic or optical technology. In such implementations, the software presented herein can transform the physical state of magnetic or optical media, when the software is encoded therein. These transformations can include altering the magnetic characteristics of particular locations within given magnetic media. These transformations can also include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations take place in the computer 500 to store and execute software components and functionalities presented herein. It also should be appreciated that the architecture shown in FIG. 5 for the computer 500, or a similar architecture, can be utilized to implement many types of computing devices such as desktop computers, notebook computers, servers, supercomputers, gaming devices, tablet computers, and other types of computing devices known to those skilled in the art. It is also contemplated that the computer 500 might not include all of the components shown in FIG. 5, can include other components that are not explicitly shown in FIG. 5, or can utilize an architecture completely different than that shown in FIG. 5.

### ILLUSTRATIVE EMBODIMENTS

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A method of tagging an item (102) with an anti-counterfeit tag (100), the method comprising: synthesizing a plurality of synthetic polynucleotides (104) comprising random sequences (200); sequencing at least a portion of the plurality of synthetic polynucleotides (104) to obtain a plurality of original sequences (106); registering, in an electronic record (108), the original sequences (106) and a description of the item; and applying at least a portion of the plurality of synthetic polynucleotides (104) to the item (102).

Clause 2. The method of clause 1, further comprising: collecting at least a portion of the synthetic polynucleotides (104) from the item (102); sequencing at least a portion of the synthetic polynucleotides (104) collected from the item (102) to obtain a plurality of retrieved sequences (116); and determining that the item is authentic based on comparison of the plurality of the retrieved sequences (116) to the plurality of original sequences (106) in the electronic record (108).

Clause 3. The method of clause 2, wherein: sequencing at least a portion of the synthetic polynucleotides collected from the item comprises sequencing fewer than all of the plurality of synthetic polynucleotides collected from the item, wherein a size of the portion is based on a value of the item; and determining that the item is authentic comprises determining that the retrieved sequences have at least a threshold level of similarity to sequences included in the original sequences.

Clause 4. The method of clause 2 or 3, further comprising: providing the plurality of retrieved sequences (116) to a computing device (110) communicatively connected to the electronic record (108); and receiving from the computing device (110) an indication of authenticity (118).

Clause 5. The method of any of clauses 1-4, further comprising: taking one or more random subsets (204) of the plurality of synthetic polynucleotides (104) prior to the sequencing and the portion of the plurality of synthetic polynucleotides that are sequenced are the synthetic polynucleotides in the one or more random subsets (204).

Clause 6. The method of clause 5, further comprising: taking two or more of the random subsets of the plurality of synthetic polynucleotides; and assembling randomly selected synthetic polynucleotides from the two or more of the random subsets of the plurality of synthetic polynucleotides to generate a plurality of assembled polynucleotides (206), wherein sequencing at least a subset of the plurality of synthetic polynucleotides comprises sequencing the plurality of assembled polynucleotides (206).

Clause 7. The method of clause 6, wherein a number of the one or more random subsets (204) used for assembly of the assembled polynucleotides (206) is based on a value of the item (102).

Clause 8. The method of clause 6 or 7, wherein the assembling is performed by Gibson assembly, Overlap-Extension Polymerase Chain Reaction, or Golden Gate assembly.

Clause 9. The method of any of clauses 6-8, wherein the assembled polynucleotides (206) are assembled from three or more random subsets (204), the synthetic polynucleotides (104) in each of the three or more random subsets (204) include non-random end sequences (202) different from the non-random end sequences (202) in the other of the three or more random subsets (204), and an order of assembly of the synthetic polynucleotides (102) from the three or more random subsets (204) is specified by the end sequences (202).

Clause 10. The method of any of clauses 1-9, wherein the plurality of synthetic polynucleotides (102) is synthesized by enzymatic synthesis and an average length of the synthetic polynucleotides is greater than 400 nucleotides.

Clause 11. The method of clause 1, wherein: sequencing the synthetic polynucleotides collected from the item comprises sequencing a portion of the synthetic polynucleotides; and determining that the item is authentic comprises identifying the sequences of the portion of the plurality of synthetic polynucleotides in the plurality of original sequences.

Clause 12. The method of any of clauses 1-10, wherein individual ones of the plurality of synthetic polynucleotides comprises a forward primer binding site and a reverse primer binding site that are not random.

Clause 13. The method of clause 12, wherein the forward primer binding site and the reverse primer binding site are both positioned between two random sequences.

Clause 14. The method of any of clauses 1-13, wherein the electronic record is maintained on one or more network-accessible computing devices at one or more locations physically distant from the item.

Clause 15. A method of tagging an item with an anti-counterfeit tag, the method comprising: synthesizing a plurality of synthetic polynucleotides (104) comprising random sequences (200); taking a first random subset (204A) and a second random subset (204B) of the plurality of synthetic polynucleotides (104); assembling randomly selected synthetic polynucleotides from the first random subset (204A) and the second random subset (204B) to generate a plurality of assembled polynucleotides (206); sequencing the plurality of assembled polynucleotides (206) to obtain a plurality of original sequences (106); registering, in an electronic record (108), the plurality of original sequences (106) and a description of the item; and applying the plurality of assembled polynucleotides (206) to the item (102).

Clause 16. The method of clause 15, further comprising: collecting at least a portion the plurality of assembled polynucleotides from the item; sequencing at least a portion of the plurality of assembled polynucleotides collected from the item to obtain a plurality of retrieved sequences; and determining that the item is authentic based on comparison of the plurality of the retrieved sequences to the plurality of original sequences in the electronic record.

Clause 17. An item (102) labeled with an anti-counterfeit tag (100), wherein the anti-counterfeit tag (100) is a plurality of synthetic polynucleotides (102) comprising random sequences (200) and the random sequences of the plurality of synthetic polynucleotides are uniquely associated in an electronic record (108) with a description of the item (302) thereby indicating authenticity of the item (102).

Clause 18. The item of clause 17, wherein each of the plurality of synthetic polynucleotides have the same forward and reverse primer binding sites.

Clause 19. The item of clause 17 or 18, wherein the plurality of synthetic polynucleotides are synthesized by column synthesis and a number of the plurality of synthetic polynucleotides with unique random sequences at least 10¹² polynucleotides.

Clause 20. The item of any of clauses 17-19, wherein the plurality of synthetic polynucleotides are assembled polynucleotides (206) comprising at least two random sequences (200) separated by two end sequences (202) that are not random.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of ±10% of the stated value.

For ease of understanding, the processes discussed in this disclosure are delineated as separate operations represented as independent blocks. However, these separately delineated operations should not be construed as necessarily order-dependent in their performance. The order in which the processes are described is not intended to be construed as a limitation, and unless otherwise contradicted by context any number of the described process blocks may be combined in any order to implement the process or an alternate process. Moreover, it is also possible that one or more of the provided operations is modified or omitted.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A method of tagging an item with an anti-counterfeit tag, the method comprising:
synthesizing a plurality of synthetic polynucleotides comprising random sequences;
sequencing at least a portion of the plurality of synthetic polynucleotides to obtain a plurality of original sequences;
registering, in an electronic record, the original sequences and a description of the item; and
applying at least a portion of the plurality of synthetic polynucleotides to the item.

2. The method of claim 1, further comprising:
collecting at least a portion of the synthetic polynucleotides from the item;
sequencing at least a portion of the synthetic polynucleotides collected from the item to obtain a plurality of retrieved sequences; and
determining that the item is authentic based on comparison of the plurality of the retrieved sequences to the plurality of original sequences in the electronic record.

3. The method of claim 2, wherein:
sequencing at least a portion of the synthetic polynucleotides collected from the item comprises sequencing fewer than all of the plurality of synthetic polynucleotides collected from the item, wherein a size of the portion is based on a value of the item; and
determining that the item is authentic comprises determining that the retrieved sequences have at least a threshold level of similarity to sequences included in the original sequences.

4. The method of claim 2, further comprising:
providing the plurality of retrieved sequences to a computing device communicatively connected to the electronic record; and
receiving from the computing device an indication of authenticity.

5. The method of claim 1, further comprising:
taking one or more random subsets of the plurality of synthetic polynucleotides prior to the sequencing and the portion of the plurality of synthetic polynucleotides that are sequenced are the synthetic polynucleotides in the one or more random subsets.

6. The method of claim 5, further comprising:
taking two or more of the random subsets of the plurality of synthetic polynucleotides; and
assembling randomly selected synthetic polynucleotides from the two or more of the random subsets of the plurality of synthetic polynucleotides to generate a plurality of assembled polynucleotides, wherein sequencing at least a subset of the plurality of synthetic polynucleotides comprises sequencing the plurality of assembled polynucleotides.

7. The method of claim 6, wherein a number of the one or more random subsets used for assembly of the assembled polynucleotides is based on a value of the item.

8. The method of claim 6, wherein the assembling is performed by Gibson assembly, Overlap-Extension Polymerase Chain Reaction, or Golden Gate assembly.

9. The method of claim 6, wherein the assembled polynucleotides are assembled from three or more random subsets, the synthetic polynucleotides in each of the three or more random subsets include non-random end sequences different from the non-random end sequences in the other of the three or more random subsets, and an order of assembly of the synthetic polynucleotides from the three or more random subsets is specified by the end sequences.

10. The method of claim 1, wherein the plurality of synthetic polynucleotides is synthesized by enzymatic synthesis and an average length of the synthetic polynucleotides is greater than 400 nucleotides.

11. The method of claim 1, wherein:
sequencing the synthetic polynucleotides collected from the item comprises sequencing a portion of the synthetic polynucleotides; and
determining that the item is authentic comprises identifying the sequences of the portion of the plurality of synthetic polynucleotides in the plurality of original sequences.

12. The method of claim 1, wherein individual ones of the plurality of synthetic polynucleotides comprises a forward primer binding site and a reverse primer binding site that are not random.

13. The method of claim 12, wherein the forward primer binding site and the reverse primer binding site are both positioned between two random sequences.

14. The method of claim 1, wherein the electronic record is maintained on one or more network-accessible computing devices at one or more locations physically distant from the item.

15. A method of tagging an item with an anti-counterfeit tag, the method comprising:
synthesizing a plurality of synthetic polynucleotides comprising random sequences;
taking a first random subset and a second random subset of the plurality of synthetic polynucleotides;
assembling randomly selected synthetic polynucleotides from the first random subset and the second random subset to generate a plurality of assembled polynucleotides;
sequencing the plurality of assembled polynucleotides to obtain a plurality of original sequences;
registering, in an electronic record, the plurality of original sequences and a description of the item; and
applying the plurality of assembled polynucleotides to the item.

16. The method of claim 15, further comprising:
collecting at least a portion the plurality of assembled polynucleotides from the item;
sequencing at least a portion of the plurality of assembled polynucleotides collected from the item to obtain a plurality of retrieved sequences; and
determining that the item is authentic based on comparison of the plurality of the retrieved sequences to the plurality of original sequences in the electronic record.

17. An item labeled with an anti-counterfeit tag, wherein the anti-counterfeit tag is a plurality of synthetic polynucleotides comprising random sequences and the random sequences of the plurality of synthetic polynucleotides are uniquely associated in an electronic record with a description of the item thereby indicating authenticity of the item.

18. The item of claim 17, wherein each of the plurality of synthetic polynucleotides have the same forward and reverse primer binding sites.

19. The item of claim 17, wherein the plurality of synthetic polynucleotides are synthesized by column synthesis and a number of the plurality of synthetic polynucleotides with unique random sequences at least 10¹² polynucleotides.

20. The item of claim 17, wherein the plurality of synthetic polynucleotides are assembled polynucleotides comprising at least two random sequences separated by two end sequences that are not random.
